(19) United States
(12) Patent Application Publication
(10) Pub. No.: US 2004/0045048 A1
(43) Pub. Date: Mar. 4, 2004

(54) Method for plant gene transferring by micro-vibration and ovary injection

(76) Inventor: Pan-Chi Liou, Taichung (TW)

Correspondence Address: CHARLES E. BAXLEY, ESQUIRE, Hart, Baxley, Daniels & Holton, 5th Fl., 59 John St., New York, NY, 10038, US

(21) Appl. No.: 10/228,366

(22) Filed: Aug. 27, 2002

(51) Int. Cl. C12N 15/82; C12N 15/87

(52) U.S. Cl. 800/278; 435/468

(57) ABSTRACT

A newly invented method for plant gene transfer by ovary injection and micro-vibration is described. First the stigma is cut off at a suitable interval of time after pollination. A tiny DNA passage is then defined by penetrating the injection needle from the position of the cut-off at the style and toward the locule inside the ovary, or further penetrating through the ovary wall near the receptacle. The exotic DNA (deoxyribonucleic acid) containing gene(s) to be transferred is injected into the locule via the passage. After injection, the ovule inside the ovary is surrounded by the exotic DNA in the locule. The ovary is then merged in water which is vibrated by a micro-vibration treatment. The micro-vibration can be created by ultra-sonic device. Under good timing management, the micro-vibration treatment will enable the injected DNA to have higher ability to enter the ovule and encounter with the fertilizing egg. This will increase the possibility of DNA recombination, i.e. gene transfer, between exotic DNA and the fertilized egg's chromosome. The transgenic fertilized egg will then naturally develop into a transgenic seed. Finally a whole transgenic plant can be obtained from a transgenic seed through germination and vegetative growth. The whole procedures are simple and easy to do, and does not need the aid of tissue culture. The method in this invention has high value and potential for practical application, since it is very simple, inexpensive, with high efficiency in plant's genetic improvement, and benefiting the environmental safety.

### FIELD OF THE INVENTION

The present invention relates to a technique about gene transferring, and more particularly, to a method for plant gene transferring by micro-vibration and ovary injection.

### BACKGROUND OF THE INVENTION

A conventional way of plant gene transferring includes the following steps: (1) cloning the gene or genes from proper sources and recombining the cloned gene(s) with proper DNA vector (plasmid) used as a gene carrier; (2) transferring part of the carrier harboring the cloned gene(s) into plant cells by proper methods so as to form transgenic cells, including particle gun bombardment method, Agrobacterium-mediated method, microinjection, electroporation, virus-mediated method, and PEG method (polyethylene glycol method) etc.; (3) the above mentioned methods all require the aid of plant tissue culture to transfer the transgenic cells by way of regeneration into transgenic plants having roots, stems, and leaves; (4) examining and screening for the successful and good transgenic plants. The above mentioned methods all have their own characteristics, however, they also have some common shortcomings which are not ready for being overcome. One of the shortcomings is that the efficiency is not always satisfied. Usually a transgenic process is more likely to be successful, if a tissue culture technique with high regeneration efficiency is available. Unfortunately, in many of the cases the tissue culture technique has not yet been well established so far. This is one of the main reasons making the success of gene transferring so rare in many crops. From this point of view, a new approach of transgenic process being able to enhance transferring efficiency and not relying on the aid of tissue culture will be very valuable.

### SUMMARY OF THE INVENTION

The object of the present invention is achieved by (1) At a suitable interval of time after pollination, injecting the DNA containing exotic gene(s) into the locule of plant ovary according to a series of operation steps to let DNA surround the ovule(s) inside the locule; (2) immediately merging the entire ovary into water and vibrating the water by ultrasonic wave; the vibration increases movement of the injected DNA around the ovule(s) so as to increase the opportunity of gene transfer in the fertilized egg.

The present invention will become more obvious from the following description when connected with the accompanying drawings which show, for purposes of illustration only, a preferred embodiment in accordance with the present invention.

### BRIEF DESCRIPTION OF THE DRAWINGS

FIG. 1 is a flow chart of the steps of the method of the present invention;

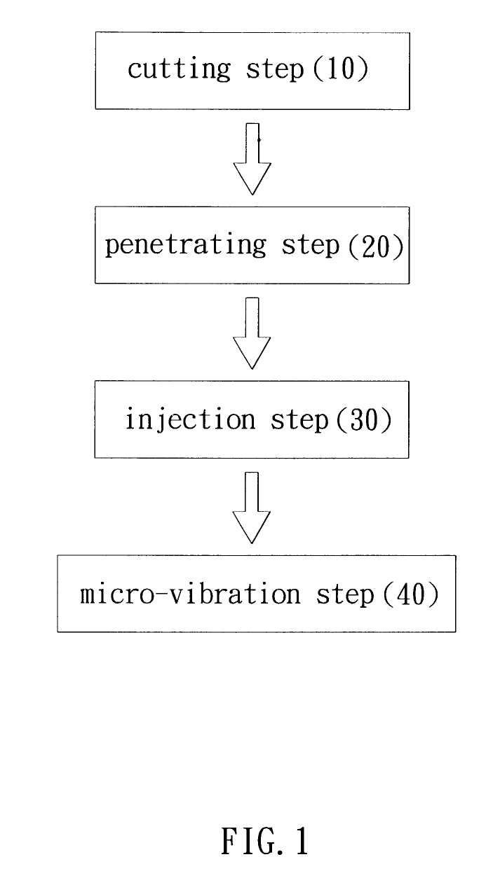

FIG. 2 shows parts of a flower of a plant, and

FIG. 3 shows the steps of cutting, penetrating, and injection;

FIG. 4 shows another way of the step of the penetrating; and

FIG. 5 shows the step of micro-vibration of the present invention.

### DETAILED DESCRIPTION OF THE PREFERRED EMBODIMENT

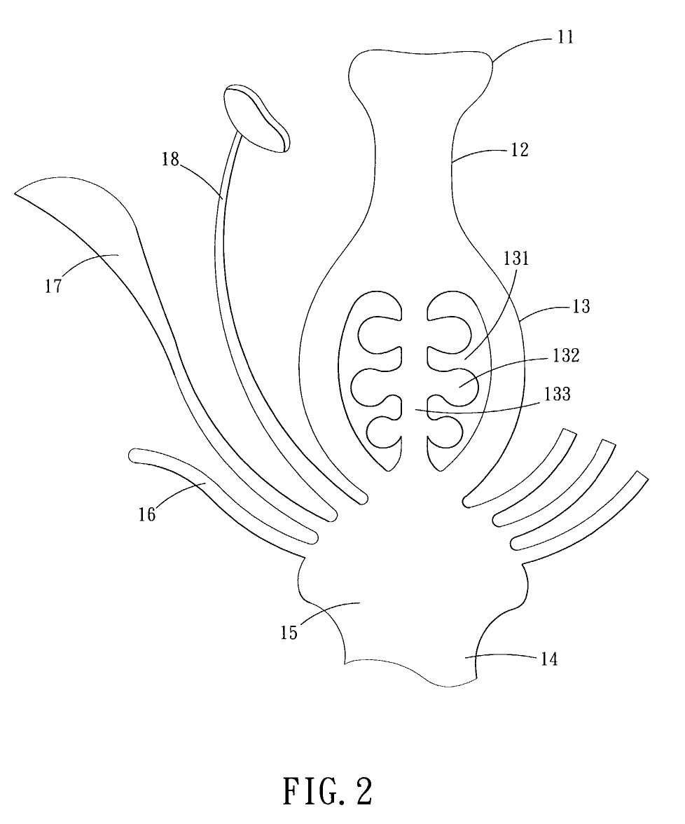

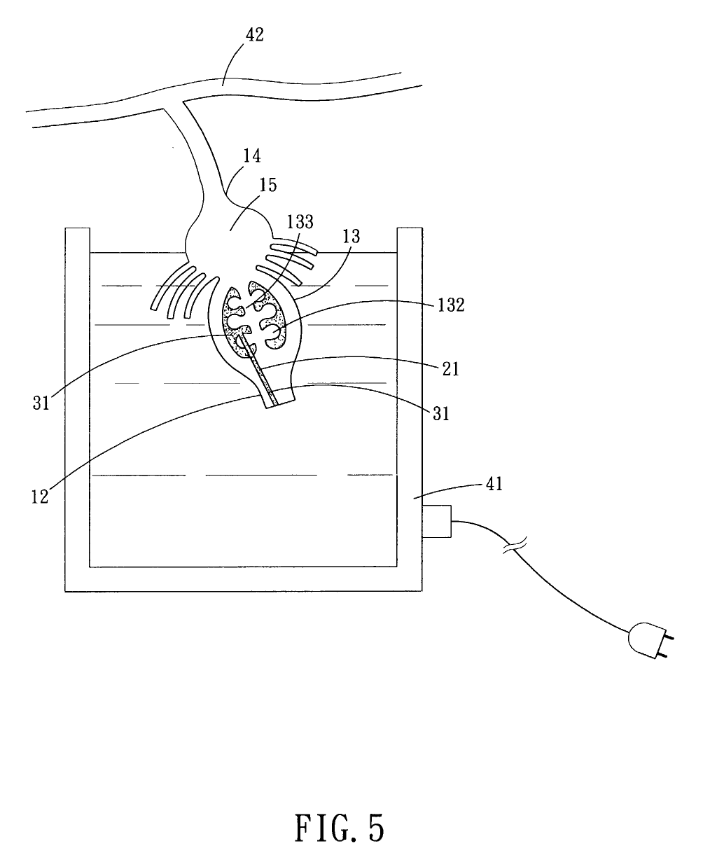

Referring to FIGS. 2 to 5, the method for transferring gene of plant of the present invention comprises four steps which are cutting step 10: cutting the stigma off at a suitable interval of time after pollination; penetrating step 20: making a tiny passage or tunnel by using an injection needle to penetrate from style toward locule; injection step 30: injecting the DNA from outside into the locule of ovary via the passage; micro-vibration step 40: merging the ovary into water and micro vibrating the water for a period of time. The four steps are operated continuously.

For the cutting step 10, referring to FIG. 2 which shows the flower of plant and includes stigma 11, style 12, ovary 13, locule 131, ovule 132, placenta 133, floral stalk 14, receptacle 15, calyx 16, petal 17, and stamen 18. After being pollinated, when pollen tubes reach the ovule(s), using a certain tool to cut off the stigma 11 at the position of the style 12 as shown.

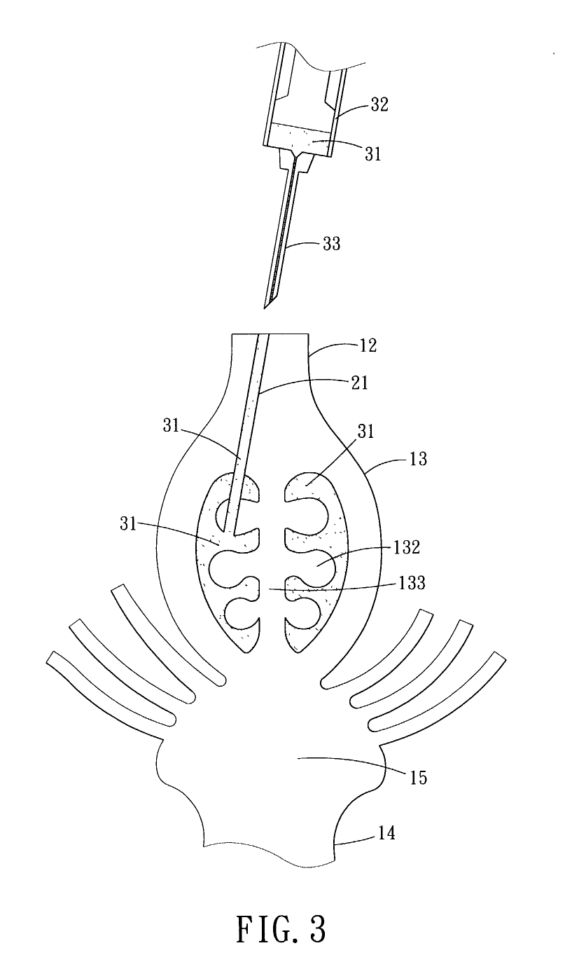

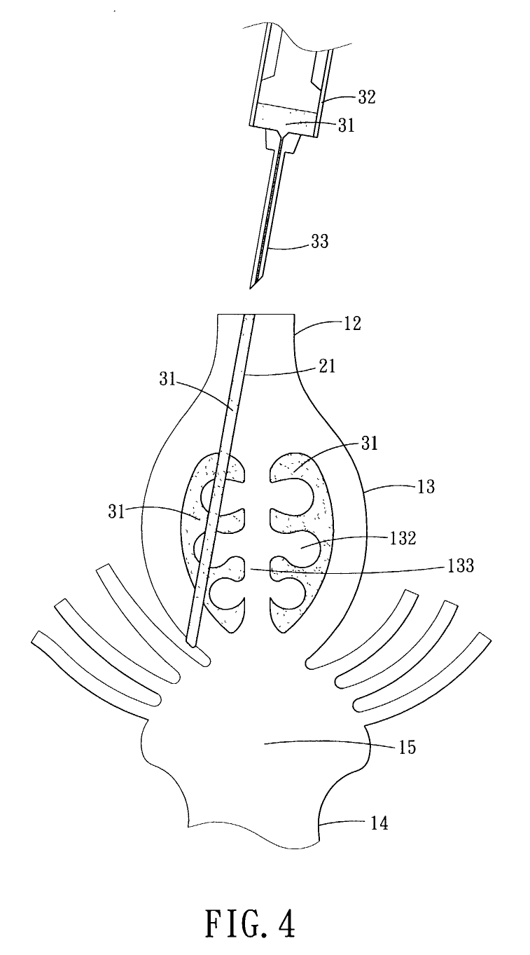

For the penetrating step 20, a tiny passage 21 is defined between the style 12 and the locule 131 by using an injection needle to penetrate into the cutting place of the style 12 and toward the ovary 13 till the locule as shown in FIG. 3, or going further through the locule 131 to penetrate the ovary wall and make an opening 34 on it as shown in FIG. 4, and then pulling the needle out to defined the tiny passage 21.

For the injection step 30, the DNA 31 is injected into the locule 131 by a needle 33 of the injection device 32 via the passage 21 to allow the DNA 31 to surround the ovule 132. If the passage 21 is defined by penetration as shown in FIG. 4, some DNA 31 could flow out from the opening 34, but this does not interrupt the result of gene transfer according to repeated tests.

For micro-vibration step 40, taking a container 41 with which water is filled and adjusting the orientation of the spray or vine 42 to adjust the flower facing downward (the ovary 13 on the top and the style 12 on the bottom) so that the ovary 13 is merged in the water inside the container 41 (the whole flower can be merged into the water to let the ovary 13 be completely surrounded with water). Micro-vibrating the water for a suitable period of time to increase the opportunity of transferring the DNA 31 into the egg inside the ovule during fertilization process. The micro vibration can be made by ultrasonic treatment.

By micro-vibration, the DNA 31 has higher possibility to enter the ovule 132 during the process of fertilization. Besides, around the time of fertilization, part of the egg's cell wall is thin and the egg cell is somewhat similar to a protoplast. This characteristic makes the DNA 31 easy to enter the egg, especially at the moment when the egg-sperm fusion (i.e. fertilization) occurs. The treatment of micro-vibration also enhances the possibility to a greater degree. As a result, the opportunity of recombination between exotic DNA 31 and the fertilized egg's chromosomes, i.e. gene transferring, is also increased. The ovule 132 containing the transgenic fertilized egg is able to form a transgenic seed through natural development afterwards, and then a complete transgenic plant can be derived from the transgenic seed without the aid of tissue culture.

Obviously, in this method the occurrence of gene transfer depends on 5 main factors including: 1. the existence of injected DNA 31 which contains exotic gene(s); 2. sperm cells traveling toward the egg inside the extending pollen tubes after pollination; 3. egg cell(s) inside ovule(s) 132; 4. the occurrence of egg-sperm fusion (i.e. fertilization) inside ovule; and 5. micro-vibration treatment which increases the movement of DNA 31. To manage all these factors in order to make gene transfer being able to occur with higher possibility, an accurate management of timing is very important for all the steps in the method of the present invention.

The present invention not only achieve the purpose of gene transfer, but also includes the following five advantages:

1. Increase the rate of success of plant gene transfer: The DNA is injected into the ovary at a suitable interval of time after pollination and then treated by micro-vibration to dramatically increase the rate of success of gene transfer.

2. Reduce the cost of performance in the research of genetic engineering: The present invention adopts the natural way of plant breeding process to achieve the purpose of gene transfer. It is simple and easy to do. Besides, it is found that using this method the efficiency is higher than the conventional ways used to transfer gene(s) into plants. This method does not need the aid of tissue culture to produce a complete transgenic plant from a transgenic cell. Such a characteristics is quite valuable for time and expenses saving, especially for those plant species in which the technique of tissue culture has not yet been well established.

3. Benefit the environmental safety: The method of the present invention neither needs tissue culture technique nor relies on bacterium mediation in all of the gene transfer process, therefore the problem of environmental contamination and pollution can be greatly reduced. There is also no damage to the eco-system, even the working procedures of this invention are all performed in the open field.

4. Easy to practice and perform: Using the method of the present invention, the practice and skill of gene transfer is easy to learn and teach. Once the technique for a specific plant was well determined and established including the timing of injection, frequency and strength of micro-vibration, and the period of time for micro-vibration treatment, the experiment of gene transfer could become a routine work and easily performed by an assistant who is even without the background of genetics and molecular biology. The main part of experiment work can be done in the field or green houses simply using a syringe of suitable size (or an injection device originally designed for gas chromatography analysis) and a micro-vibration inducing device. No expensive instrument and equipment is needed.

5. Particularly suitable and useful for those plants having multiple seeds produced in one fruit, if the fruit is originated from one single pollinated flower such as tomato and watermelon: Since ovary is the basic unit to deal with in this method, this method is especially useful and valuable for those plants with multiple seeds produced in one single-flower-originated-fruit. Apparently the rate of success can be enhanced, if multiple seeds can be obtained from one single experimental treatment. The more the seeds can be obtained from one treated ovary (flower), the higher the rate of success can be expected. In agriculture, there are many crops bear multiple-seeds-containing fruits which are developed from single pollinated flowers. Their species distribute in different taxonomic families, including Orchidaceae, Cucurbitaceae, Leguminosae, Solanaceae, Rosaceae, Cruciferae, Rutaceae, Myrtaceae, Liliaceae, Passifloracae, Oxalidaceae, Vitaceae, Actinidiaceae, and Caricaceae etc. We estimate that ca. 2/3 of horticultural crops are of this type, and most of these crops still lack good tissue culture research. In fact tissue culture research even has not yet been initiated in many of these crops, and so does the genetic engineering work, therefore, the method of this invention is going to have a huge potential for application in many crops' genetic improvement.

While we have shown and described the embodiment in accordance with the present invention, it should be clear to those skilled in the art that further embodiments may be made without departing from the scope of the present invention.

What is claimed is:

1. A method for plant gene transferring by micro-vibration and ovary injection, wherein the stigma is cut off after the flower is pollinated and pollen tubes have properly developed, a passage is defined from the style to the locule, exotic DNA is then injected via the passage into ovary to fill in the locule, the ovary is proceeded by treatment of a micro vibrating device to increase the movement of exotic DNA so as to increase the possibility for it to enter the ovule and more further enter the fertilizing egg when egg-sperm fusion occurs, and to increase the possibility of DNA recombination between exotic DNA and egg/sperm's chromosome (i.e. gene transfer).

2. The method as claimed in claim 1, wherein the micro vibration can be done by using ultrasonic treatment.

3. The method as claimed in claim 1, wherein the passage is defined by penetrating an injection needle from the position of cut-off of the style toward the locule inside the ovary, and the needle is then pulled out.

4. The method as claimed in claim 1, wherein the passage is defined by penetrating an injection needle from the position of cut-off at the style and toward the locule inside the ovary and penetrating all the way through the ovary wall near the receptacle, and the needle is then pulled out.
